(19) United States
(12) Patent Application Publication
(10) Pub. No.: US 2020/0128932 A1
(43) Pub. Date: Apr. 30, 2020

(54) TREATMENT CONTAINER FOR ACCOMMODATING AN OPHTHALMIC LENS AND CONTAINER PLATE

(71) Applicant: ALCON INC., Fribourg (CH)

(72) Inventors: Harald Bothe, Niedernhausen (DE); Felix Brinckmann, Rossdorf (DE); Alfred Fischer, Niedernberg (DE); Joachim Jung, Mombris (DE); Nils Schweizer, Bad Konig (DE); Sebastian Wedekind, Frankfurt (DE)

(21) Appl. No.: 16/668,309

(22) Filed: Oct. 30, 2019

Related U.S. Application Data
(60) Provisional application No. 62/753,346, filed on Oct. 31, 2018.

(51) Int. Cl. A45C 11/00; A61L 12/08

(52) U.S. Cl. CPC A45C 11/005; A61L 12/086

(57) ABSTRACT

A treatment container (1) for accommodating an ophthalmic lens (2) comprises a bottom (3), a side wall (4) surrounding the bottom (3) and together with the bottom (3) forming a cavity (5) for accommodating the ophthalmic lens, a container opening (6) arranged opposite to the bottom (3), and a fluid opening (7) arranged in the bottom (3) or in the side wall (4) of the treatment container (1). The fluid opening (7) is in fluid communication with the cavity (5). The treatment container (1) further comprises a fluid conveyance channel (8) and a supply connector (9). The fluid conveyance channel (8) is connected to the fluid opening (7) so as to be in fluid communication with the fluid opening (7). The fluid conveyance channel (8) is further connected to the supply connector (9) so as to be in fluid communication with the supply connector (9), to allow for introducing a liquid into the cavity (5) or for removing a treatment liquid from the cavity (5) through the supply connector (9), the conveyance channel (8) and the fluid opening (7).

This application claims the benefit under 35 USC § 119 (e) of U.S. provisional application Ser. No. 62/753,346 filed Oct. 31, 2018, incorporated herein by reference in its entirety.

### FIELD

The present invention relates to a treatment container for accommodating an ophthalmic lens, in particular a contact lens such as a hard or soft contact lens, or an intra-ocular lens, as well as to a container plate comprising a plurality of such treatment containers. The treatment container and container plate are particular useful for a coating process and for other processes of treatment of a contact lens, particularly in an automated contact lens manufacturing line.

### BACKGROUND

It is well known in the art to produce ophthalmic lenses such as contact lenses, in particular soft contact lenses, in an automated production process by using either re-usable or disposable molds. The contact lens materials and the high requirements for medical devices frequently makes it necessary that the contact lens, after being formed, is to be additionally exposed to further treatment steps before it can be placed in a package and distributed to the customer. For example, it may be necessary to immerse the ophthalmic lenses in extraction baths, rinsing baths, coating solutions and/or loading solutions in order to obtain a contact lens which may be distributed to the customer. This is because contact lenses have to have certain properties to allow them to be worn directly on the wearer's eye. For example, high oxygen permeability of the lens to maintain good corneal health is typically associated with hydrophobicity, so that the contact lens would adhere to the eye. Hence, it is necessary to have at least one surface coating or surface treatment step in order to increase the hydrophilic properties of the surface of the contact lens such that the lens is allowed to move on the wearer's eye without binding excessive amounts of tear lipid and protein.

In order to modify the hydrophilicity of the surface of the contact lens, a coating may be applied onto the surface of the contact lens, particularly using a layer-by-layer polymer adsorption (LbL) process. By iteratively immersing one or more lenses in an alternating fashion in a polyanion (e.g. polyacrylic acid, PAA) and a polycation (polyallylamine hydrochloride, PAH) solution, a LbL coating can be formed on the surface of the contact lens.

Alternatively, soft contact lenses which comprise a polyvinylalcohol-based hydrogel lens body may be treated for enhanced surface lubricity compared to the lubricity of the polyvinylalcohol-based hydrogel lens body, for example by a coating covalently attached onto the polyvinylalcohol-based hydrogel lens body.

During these process steps, it must be ensured that the entire contact lens is immersed in the treatment liquid, such that all surfaces of the contact lens are in contact with the treatment liquid. Typically, the contact lenses are transported through a number of subsequent large volume dipping baths while being accommodated in lens-carrying containers from which the lenses cannot escape and while allowing the flow of treatment liquid into and out of the lens-carrying containers. For example, the lens-carrying containers may comprise male and female basket halves which are arranged on a tray in order to allow a plurality of lens-carrying containers with contact lenses to be simultaneously dipped into the baths.

However, this method only allows for all contact lenses of all lens-carrying containers of a tray to be immersed in and hence treated by the same treatment liquid. Also, treatment times are fixed while being transported though the respective bath. Additionally, contamination of the baths may occur by treatment liquid of a preceding dipping bath which may adhere to the contact lens and/or to the lens-carrying container, and which may soil the subsequent dipping bath. Hence, contamination may increase over time with increasing cycle numbers over extended periods of time. Dipping bath volume has been increased to buffer the contamination, and laborious and expensive methods have been implemented to keep contamination of the dipping baths at low level, leading to high amounts of waste treatment liquids to be discarded. Nevertheless, even with elaborated monitoring methods for keeping contamination levels low, a constant low contamination level in the dipping baths is very difficult to maintain.

It is therefore an object of the present invention to overcome the afore-discussed disadvantages of the prior art. Another object of the invention is to provide a container and a method for treating ophthalmic lenses, for example contact lenses like soft or hard contact lenses, or intra-ocular lenses, with high flexibility, which can be readily integrated into existing production lines while enhancing handling safety and hence reducing the necessary safety measures.

### SUMMARY

These and still further objects are met by a treatment container according to the invention, by a container plate, and by a container tray according to the invention. These are particularly useful for accommodating an ophthalmic lens during its treatment by treatment solutions such as rinsing solutions, extracting solutions, coating solutions. The objects are also met by a respective method for treatment of an ophthalmic lens according to the invention.

Throughout the entire specification including the appended claims, the singular forms “a”, “an”, and “the” include the plural, unless the context explicitly dictates otherwise. Also, whenever features are combined with the term “or”, the term “or” is to be understood to also include “and” unless it is evident from the specification that the term “or” must be understood as being exclusive.

A treatment step is the single step of treating an ophthalmic lens with a specific treatment liquid, whereas a treatment cycle comprises the totality of the treatment steps the ophthalmic lens is subjected to. Also, the treatment time is the contact time of the ophthalmic lens with the liquid during the respective treatment step, whereas the cycle time is the total time of the treatment cycle.

To achieve the above-mentioned objects, the present invention suggests a treatment container as it is specified by the features of the independent claim. Advantageous aspects of the container according to the invention are the subject of the dependent claims directed to the container.

In particular, the present invention suggests a treatment container for accommodating an ophthalmic lens, in particular a contact lens such as a hard or soft contact lens, or an intraocular lens. The treatment container comprises a bottom, a side wall surrounding the bottom and together with the bottom forming a cavity for accommodating the ophthalmic lens. The treatment container further comprises a container opening arranged opposite to the bottom, and a fluid opening arranged in the bottom or in the side wall of the treatment container. The fluid opening is in fluid communication with the cavity. The treatment container further comprises a fluid conveyance channel and a supply connector. The fluid conveyance channel is connected to the fluid opening so as to be in fluid communication with the fluid opening, and is further connected to the supply connector so as to be in fluid communication with the supply connector, to allow for introducing a treatment liquid into the cavity or for removing a treatment liquid from the cavity through the supply connector, the conveyance channel and the fluid opening.

According to one aspect of the treatment container according to the invention, a surface of the bottom that faces towards the cavity is concavely shaped.

According to a further aspect of the treatment container according to the invention, the treatment container has a rim at one end of the side wall arranged at the container opening. The rim is arranged in a plane and surrounds the container opening. The fluid opening is arranged, at least in part, in the bottom of the container at a location in the cavity which is arranged most distally from the plane in which the rim surrounding the container opening is arranged.

According to a further aspect of the treatment container according to the invention, a surface of the bottom that faces towards the cavity comprises a structure facing the interior of the cavity, to allow for uniform wetting of the ophthalmic lens.

According to still a further aspect of the treatment container according to the invention, the structure comprises one or more grooves arranged in the bottom surface, in particular four grooves arranged at 90° or eight grooves arranged at 45° relative to one another when viewed in a circumferential direction of the container. The grooves meet at an intersection located centrally in the bottom surface.

According to yet another aspect of the treatment container according to the invention, the fluid opening is connected, at least in part, to one of the one or more grooves arranged in the bottom surface so as to be in fluid communication with the one of the one or more grooves, to allow for introduction and/or removal of the treatment liquid.

Another aspect of the invention relates to a container plate comprising a plurality of treatment containers according to the invention.

In accordance with one aspect of the container plate according to the invention, the supply connectors of two or more treatment containers of the plurality of treatment containers together form a common supply connector, and the fluid openings of the two or more treatment containers are in fluid communication with the common supply connector via the conveyance channels of the two or more treatment containers, for concurrently introducing a treatment liquid into or concurrently removing a treatment liquid from the two or more treatment containers which are in fluid communication with the common supply connector.

In accordance with a further aspect of the container plate according to the invention, the common supply connector is configured to receive a supply pipe, in particular a suction pipe from a liquid aspirator.

According to a further aspect of the container plate according to the present invention, the container plate has a plate bottom and a plate top surface, and each of the supply connectors has a supply pipe receiving opening which is arranged at the plate top surface opposite to the plate bottom.

According to a further aspect of the container plate according to the invention, the plurality of treatment containers are integrally formed with the container plate, so that the container plate including the plurality of treatment containers is made from one piece, thus forming a monolithic container plate. However, the container plate may also be formed by more than one piece which are assembled to form the container plate.

Yet in accordance with another aspect of the container plate according to the invention, the plurality of treatment containers, the liquid conveyance channels and the supply connectors are formed during injection molding of the monolithic container plate, by a deep drawing process, or by milling a bulk plate. Optionally, a structure in the bottom surface, in particular the grooves, are also formed during this manufacturing step.

In still some additional aspect of the container plate according to the invention, the liquid conveyance channels and the supply connectors are integrated into the (monolithic) container plate and are in fluid communication with at least one of the grooves of the treatment container.

A further aspect of the invention relates to a container tray comprising a container plate according to the invention and a container plate frame holding the container plate and having fixtures adapted to cooperate with corresponding fixtures of a production facility.

An additional aspect of the invention relates to a method for treatment of an ophthalmic lens, in particular a contact lens such as a hard of soft contact lens, or an intraocular lens, with a treatment liquid.

The method comprises the steps of:
providing a container plate according to the invention,
introducing ophthalmic lenses into the cavities of the treatment containers of the container plate,
filling the treatment containers with a treatment liquid either through the container openings or through the fluid openings,
removing the treatment liquid through the fluid openings, either after a predetermined time during which the ophthalmic lenses are exposed to the treatment liquid or during filling the treatment containers with the treatment liquid,
optionally repeating the steps of filling the treatment containers with a treatment liquid and removing a treatment liquid in accordance with a predetermined treatment scheme to complete a treatment cycle, and
removing the ophthalmic lenses from the cavities of the treatment containers.

In a further aspect of the method according to the invention, the treatment containers are rinsed with a rinsing liquid, particularly deionized water, between two subsequent treatment cycles, i.e. after removal of a treated ophthalmic lenses and before introduction of the subsequent ophthalmic lenses to be treated.

The afore-mentioned embodiments are practical embodiments of treating an ophthalmic lens, in particular a hard or soft contact lens or an intra-ocular lens.

The treatment container according to the invention allows for very reproducible treatment of the ophthalmic lens in the treatment container. This leads to ophthalmic lenses of excellent quality and high reliability. Due to the very small amount of treatment liquid needed compared to a treatment bath, the treatment liquid may be freshly supplied for each treatment step. Suction of the previous treatment liquid from the treatment container reduces the amount of contamination of the subsequent treatment liquid and leads to very reproducible treatment steps in each treatment cycle of the ophthalmic lens. When the treatment steps comprise one or more coating steps, the treatment container according to the invention leads to ophthalmic lenses with very reproducible coatings of high quality. The coating is of uniform quality from one treatment cycle to the next, i.e. it is of uniform quality also for the subsequently coated ophthalmic lenses.

It is thus possible that the ophthalmic lens be exposed to several treatment liquids (including coating and cleaning liquids) one after the other while the ophthalmic lens remains in the treatment container. No handling or gripping action is required to transfer the ophthalmic lens from one container or bath to another container or bath in order to expose the ophthalmic lens to different treatment liquids.

The treatment containers also allow for customized treatment from one treatment cycle to the other. This is of particular interest for small series of ophthalmic lenses which may be treated, in particular coated, according to the needs of production. The treatment container according to the invention leads to high flexibility in the treatment of the ophthalmic lens and fast modification of the treatment scheme (nature of the treatment liquids, composition of the treatment liquids, concentration of the treatment liquids, treatment time for each treatment step), which may be individually adapted to the needs of the ophthalmic lens. With the aid of the treatment container according to the invention, very short contact time periods with the respective treatment liquid may be chosen.

As the very small volume of the treatment container is drained (sucked) between each treatment step, the composition of the subsequent treatment liquid may be adapted to the needs and may be minutely dosed to the ophthalmic lens accommodated in the treatment container.

The treatment container according to the invention also has the advantage of offering the possibility of easily subjecting the surface of the ophthalmic lens to surface tailoring chemistry which allows for modifications to the ophthalmic lens surface. This may lead, for example, to fundamental modifications of the surface and its interaction with other substances, for example in or on the wearer's eye.

Additionally, the very low volume of the treatment containers leads to small liquid holdup having an open surface towards the facility atmosphere, and hence leads to very easy handling of the liquids and higher safety levels when compared to the use of treatment baths. Hence, the necessary safety measures such as fire safety measures, for such small liquid amounts and in particular for such small liquid surfaces, can be easily met.

Furthermore, leak protection through a retention tank can be reduced, again due to the small amounts of liquids handled in the treatment containers.

Due to the very small dimensions of the treatment containers and in particular of the container plate according to the invention, high integrability into existing facilities is given with minimal integration complexity. The required infrastructure is simple and inexpensive, and no particular piping is needed. Hence, a later addition of a coating step in the facility, for example to provide the ophthalmic lens with a surface enhancement coating, can be easily performed.

When the bottom surface has a concave shape, the treatment container is better adapted to accommodate the ophthalmic lens which generally has at least one convex surface. The treatment container may be bowl-shaped or cup-shaped.

When the fluid opening is located at the lowest point of the treatment container (most distal from the rim of the container opening), the treatment liquid removal is particularly efficient as the liquid will be transported to the lowest point by gravity. However, the fluid opening does not have to be confined to the lowest point of the treatment container. Rather, the fluid opening may extend from the lowest point of the treatment container towards the container opening and through the wall of the treatment container.

The structure on or in the bottom of the surface of the treatment container enhances the uniform wetting of the ophthalmic lens, thereby ensuring complete contact of the ophthalmic lens with the treatment liquid for optimal treatment and coating processes. Additionally, such structure may support treatment liquid removal from the cavity.

One or more grooves in the bottom (an example of such structure) are particularly advantageous regarding the wetting of the ophthalmic lens and the functional ability of efficient treatment liquid removal when connected to the fluid opening. In particular four grooves or eight grooves arranged normal to one another in a star-shaped arrangement have been found to show excellent properties and are easy of manufacture. The four grooves or eight grooves meet at a centrally located point (intersection) in the bottom surface of the treatment container, such that the distance from the wall surrounding the cavity of the treatment container at the edge of the bottom surface to the point of intersection is constant.

In particular, when the grooves are combined with the concave shape of the bottom surface, this may enhance treatment liquid removal from the cavity.

A container plate comprising a plurality of treatment containers according to the invention allows for fast processing of several ophthalmic lenses accommodated in the same container plate. For example, the container plate may comprise four rows of ten treatment containers allowing for up to forty ophthalmic lenses to be brought into contact with the treatment liquids.

The container plate may be particularly made of PTFE and derivatives, PE, PP, PET, PEEK, stainless steel, anodized aluminum, etc.

The container plate according to the invention additionally allows for maximum flexibility during the lens manufacturing process. For example, when fewer ophthalmic lenses are produced, less than forty treatment containers may be loaded with ophthalmic lenses. With the aid of an adequate control system, only those treatment containers actually accommodating an ophthalmic lens may be supplied with treatment liquid. This procedure will additionally reduce treatment liquid consumption and discharge.

Moreover, each treatment container may accommodate an individual ophthalmic lens which may differ from the ophthalmic lens in the adjacent treatment container. For example, in the case of a container plate comprising forty treatment containers, up to forty different ophthalmic lenses may be accommodated in the container plate. Each treatment container may then be filled with an individually composed treatment liquid depending on the ophthalmic lens accommodated in the respective treatment container. For example, when a contact lens with high diopters is to be treated, a treatment liquid with higher concentration may be added to the corresponding treatment container if necessary.

When the fluid openings of two or more treatment containers are connected to a common supply connector—via the fluid conveyance channels of the respective individual treatment containers—the number of supply pipes is reduced accordingly. For example, when two fluid openings of two treatment containers are connected via the fluid conveyance channels with a common supply connector, optionally repeating the steps of filling the treatment container with the treatment liquid, treating the ophthalmic lens and removing the treatment liquid in accordance with a predetermined treatment scheme to complete a treatment cycle, can be performed with only one supply pipe for removing the treatment liquid from both cavities. In case of less than forty ophthalmic lenses actually accommodated by the container plate, skipping of cavities is then performed pairwise. Of course, filling of individually composed treatment liquid is then also possible for pairs of treatment containers (and cavities) only.

The supply connectors are advantageously designed to allow for reversible, detachable insertion of the (vacuum or suction) supply pipes from the liquid aspirator. The liquid aspirator is part of the facility in which the container plate is used. For simplicity, the supply connectors may have a cylindrical shape. Advantageously, the supply connectors are arranged vertically in the container plate.

The supply pipe receiving openings are particularly arranged at the plate top surface of the plate opposite to the plate bottom, so that no additional valves are necessary to prevent any treatment liquid from flowing out of the cavity due to gravity. This embodiment leads to particular ease of handling of the container plate as the supply channels are arranged vertically with the supply pipe receiving openings facing upwards, thereby avoiding any dripping of treatment liquid out of the treatment container.

When a monolithic container plate is formed (this means that the plate is formed as a single large block), the malfunction rate of the container plate is reduced to a minimum as there are no additional parts in or on the plate which may lead to the interruption of the process.

Additionally, such monolithic container plate is particularly easy to manufacture by injection molding, by a deep drawing process or a milling process. In case of a milling process, a bulk plate (large raw plate used for the manufacture of the container plate) is used, which has the dimensions of the final container plate, and the containers, grooves, fluid openings, conveyance channels and connections from the fluid openings to the conveyance channels are formed into the bulk plate by a milling process. Alternatively, the containers, grooves, fluid openings, conveyance channels and connections from the fluid openings to the conveyance channels may also be formed, at least partially, by injection molding or by a deep drawing process.

The container plate may be framed by a container plate frame holding the container plate and forming the junction between the container plate and the production facility, the container plate and the container plate frame thus forming a container tray. The frame also allows for quick substitution of the container plate, in case the container plate is damaged, or in case another treatment container material is necessary for the treatment scheme. Therefore, the treatment container according to the invention is particularly flexible in use, as the material the walls of the treatment container are made of may be adequately adapted to the liquids the treatment container will be exposed to depending on the treatment scheme to be carried out.

Using a container tray has the additional advantage to easily stack the plates in case a subsequent production step has a longer cycle time. For example, when the cycle time of the treatment of the ophthalmic lenses in the treatment containers is 5 seconds in a previous process step and the subsequent cycle time is 50 seconds in a subsequent process step, a total of ten trays may be stacked in order to match the cycle time of the subsequent process step. In this case, the tray that has been stacked the earliest is the first tray that is conveyed from the stack to the next process step, whereas a new tray coming from the previous process step is stacked at the end of the stack. This procedure may also be used to store trays in case of a downtime of one or more subsequent process step(s).

The above-discussed advantages for the specific aspects for the container, the container plate and the container tray are equally applicable to the method according to the invention.

The steps of introducing treatment liquids, treatment period and removal of the treatment liquid may be repeated if necessary according to a defined treatment scheme. In case only one treatment step is required, the treatment cycle will stop after only one treatment step.

When the treatment liquid is removed through the fluid opening during filling the treatment container with the treatment liquid, the treatment container is filled with a treatment liquid through the container opening. In this case, filling the treatment liquid and removing the treatment liquid through the fluid opening may occur concurrently to obtain a continuous flow of treatment liquid, for example for rinsing the ophthalmic lens.

Alternatively, the ophthalmic lens is treated by exposing the ophthalmic lens to the treatment liquid for a predetermined treatment time, and after the predetermined treatment time the treatment liquid is removed through the fluid opening (via the fluid conveying channel and the supply connector). The steps of filling the treatment container with a treatment liquid, treating the ophthalmic lens and removing a treatment liquid in accordance with a predetermined treatment scheme are optionally repeated to complete a treatment cycle.

Between two treatment cycles, an inspection step may be introduced to check whether all treatment containers in the container plate are empty so that the containers and the container plate are ready for the next cycle.

Further embodiments and advantages become apparent from the following description of detailed embodiments of the invention with the aid of the drawings.

It is to be noted, that each individual feature described herein as well as all combinations of two or more of such features are possible as long as such features are not mutually exclusive or are otherwise technically incompatible.

### BRIEF DESCRIPTION OF THE DRAWINGS

Further details and advantages of the invention will become apparent from the following description of exemplary embodiments of the invention with the aid of the drawings, in which:

FIG. 1 shows an embodiment of a single treatment container according to the invention;

FIG. 2 shows a portion of an embodiment of the container plate according to the invention;

FIG. 3 shows the embodiment of the container plate of FIG. 2 immediately prior to the step of removing a liquid from a treatment container of the container plate using a suction pipe;

FIG. 4 shows a partially sectional view of the container plate of FIG. 3 during removal of the liquid with the aid of the suction pipe; and

FIG. 5 shows a container tray according to the invention.

### DETAILED DESCRIPTION

As used in this specification, the term “treatment liquid” or “treatment liquids” comprises any type of liquid to which the ophthalmic lens, in particular a contact lens such as a soft contact lens or an intraocular lens, may be exposed during a contact lens manufacturing process, and include in particular liquids influencing the physical or chemical properties of the lens. Without being exhaustive, such treatment liquids may comprise extraction liquids, rinsing liquids, coating liquids or any other type of liquid, and in particular also may comprise water.

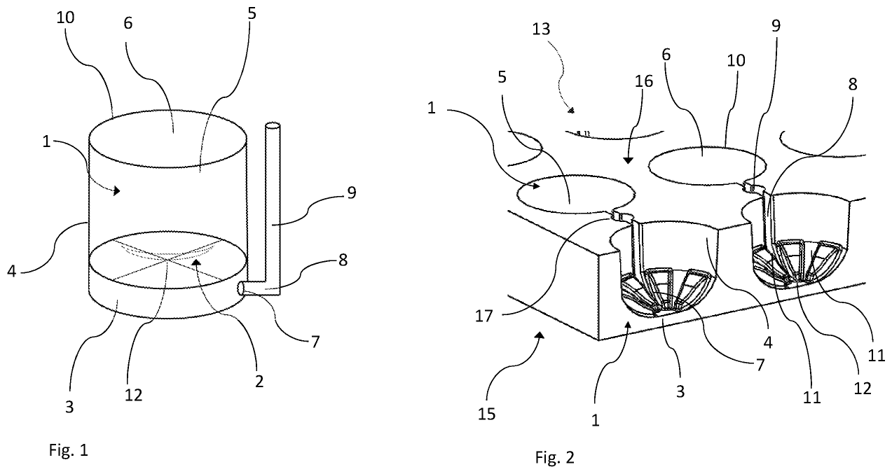

FIG. 1 shows an embodiment of a single treatment container 1 of the present invention accommodating a contact lens 2. For the following description, a contact lens 2 is an example of and represents the ophthalmic lens. The treatment container 1 may be cylindrical and has a bottom 3 on the surface of which the contact lens 2 rests, as well as a side wall 4 which—together with the bottom 3—forms a cavity 5 of the treatment container 1. The treatment container 1 has a container opening 6 arranged at the upper end of the container opposite to the bottom 3 of the treatment container 1 which is surrounded by a rim 10 formed at the upper end of the side wall 4. The contact lens 2 can be introduced or removed through the container opening 6, for example with the aid of a gripper.

The treatment container 1 additionally comprises a fluid opening 7, a fluid conveyance channel 8 and a supply connector 9 for introducing a liquid into or removing a liquid from the cavity 5. The fluid opening 7 is in fluid communication with the lowest point of the cavity 5 and with the fluid conveyance channel 8. The fluid conveyance channel 8 is connected to the supply connector 9 to which a supply pipe from the facility may be connected for supplying treatment liquid to the treatment container 1, or for supplying vacuum to remove the treatment liquid from the treatment container 1.

In the bottom surface of the treatment container 1, a star-shaped structure is embedded in the bottom surface for enhancing the uniform wetting of the contact lens 2. The individual elements of the star-shaped structure meet at a centrally located intersection 12 in the bottom surface.

Lenses can be introduced into and removed from the treatment container 1 with the aid of a conventional gripper (not shown). For introduction of a lens into the treatment container 1, the gripper with the lens attached thereto is moved downwardly into the cavity 5 of the treatment container 1 and towards the bottom 3 of the treatment container 1. Once the gripper has released the lens and has been retracted, the treatment steps may start.

Similarly, for removal of the lens from the containment element, the lens can be gripped by the gripper and can then be removed from the treatment container 1.

FIG. 2 shows a portion of an embodiment of a container plate 13 having a plurality of treatment containers 1. The bottom surfaces are convex and the cavities 5 of the treatment containers 1 are cup-shaped for optimal accommodation of the contact lens. The structure in the bottom surface of the treatment container 1 comprises eight grooves 11 which are arranged at 45° relative to one another. One of these grooves 11 is connected via the fluid conveyance channel 8 to the supply connector 9.

In the embodiment shown in FIG. 2, the monolithic container plate is made from a bulk PTFE block by a milling process. In particular the cup-shaped treatment containers 1 (concave bottom), the eight grooves 11, the fluid conveying channels 8 and the supply connector 9 of each of the treatment containers 1 are formed in the PTFE block during the milling process. The eight grooves 11 are connected to the fluid opening 7 of the respective treatment container 1 at the lowermost location of the treatment container (i.e. the fluid opening 7 is arranged a location most distally from the plane in which the rim 10 surrounding the respective container opening 6 is arranged).

The supply connector 9 has a cylindrical shape and is arranged vertically between a plate bottom 15 and a plate top surface 16 of the container plate 13. The walls of the supply connector 9 comprise two vertically arranged slots extending from the plate bottom 15 to the plate top surface 16 where a supply pipe receiving opening 17 is arranged. These slots connect the supply connector 9 with the conveyance channels 8 and with a groove 11 of each of the two adjacently arranged treatment containers 1, so that supply connector 9 forms a common supply connector for the two adjacently arranged treatment containers 1. The connection of two adjacently arranged treatment containers 1 to the common supply connector 9 allows for the concurrent supply of treatment liquid to the two adjacently arranged treatment containers 1, as well as for concurrent vacuum removal of the treatment liquid from these two containers 1.

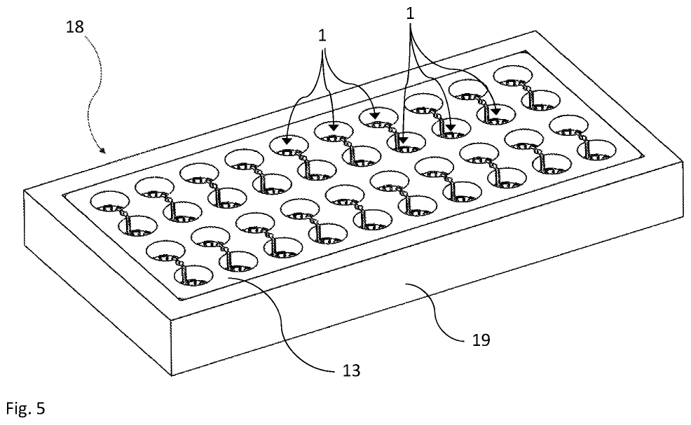

This connection of two adjacently arranged treatment containers 1 to a common supply connector 9 can be seen best when glancing at FIG. 4. One groove 11 of each of the two adjacently arranged treatment containers 1 is in fluid communication with the common supply connector 9 via the corresponding fluid conveyance channel 8. The respective groove 11 of each treatment container 1 extends through the wall of and into the vertical common supply connector 9 and serves as the fluid opening 7 of the treatment containers 1. The extension of the groove 11 through the wall and into the common supply connector 9 forms the fluid conveyance channel 8.

The set-up of the grooves and their respective extension into a supply connector may of course also be used when one treatment container is connected to only one supply connector (i.e. each container has its own supply connector). However, it may also be used in case more than two treatment containers 1 are connected to a common supply connector.

In the present embodiment, the pair-wise arrangement of treatment containers 1 connected to a common supply connector 9 is repeated over the whole container plate 13 having a total of forty treatment containers 1, so that twenty pairs of treatment containers 1 are pair-wise in fluid communication and connected to twenty common supply connectors 9. Hence, the treatment liquids may be individually composed for up to 20 pairs of contact lenses simultaneously in the corresponding treatment containers 1.

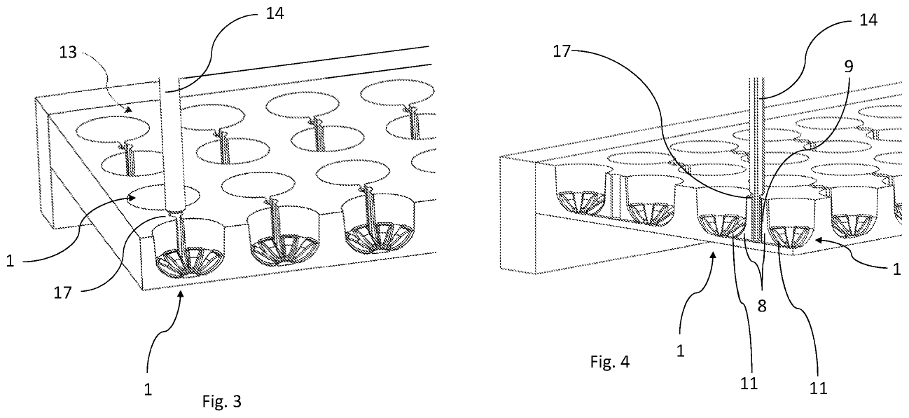

In FIG. 3 and FIG. 4, the common supply connector 9 is shown in the liquid removal mode, i.e. when a suction pipe 14 (a supply pipe supplying vacuum) from the facility is connected or about to be connected to the common supply connector 9 through the supply pipe receiving opening 17. The suction pipe 14 (see FIG. 3) is inserted through the supply pipe receiving opening 17 into the supply connector 9 until its pipe opening is located a short distance above the plate bottom 15 which also forms the bottom of the common supply connector 9 (see FIG. 4). Upon applying vacuum, the treatment liquid contained in the cavities 5 of the treatment containers 1 is allowed to flow out of the two adjacently arranged treatment containers 1 into the suction pipe 14. In this arrangement, up to twenty suction pipes 14 may be inserted into the corresponding twenty supply connectors 9 for concurrent removal of treatment liquids.

FIG. 5 shows a container tray 18 in which a container plate 13 comprising twenty pairs of treatment containers 1 (similar to FIG. 3 and FIG. 4) is framed by a container plate frame 19. The container plate frame 19 comprises fixtures which allow the container tray to cooperate with corresponding fixtures on the production facility.

In one embodiment, the method for treatment of a contact lens 2 (representing an ophthalmic lens) is as follows:

The contact lens 2 is introduced into the cavity 5 (i.e. into the container 1) by a gripper that transports the ophthalmic lens 2 from a production line to the treatment station comprising a container plate 13. Generally, a plurality of grippers simultaneously transports a plurality of ophthalmic lenses from the production line to the treatment station. For constructional design reasons, it may be advantageous to place a contact lens only into every second treatment container 1 in a first step, and to place a contact lens into the remaining (empty) treatment containers 1 in a second step. Once a contact lens has been placed into all desired treatment containers 1 the treatment cycle may start.

For this purpose, treatment liquid is introduced, with the aid of a precision dosing system, into each treatment container 1 as desired from above the container plate 13, for example through the corresponding container opening 6. The dosing system is brought in alignment with the container plate and the dosing step of the treatment liquids may proceed. Alternatively, the container plate 13 may be moved to a dosing station for the same purpose.

After a predetermined treatment time, the treatment liquid is removed through the application of suction. To this end, a plurality of suction pipes 14 connected to a liquid aspirator are moved over the container plate 13, and the individual suction pipes 14 are introduced into the corresponding supply connectors 9 of the container plate 13 through the supply pipe receiving openings 17. Alternatively, the container plate 13 may be moved to a suction station for the same purpose. The vacuum may be applied before the suction pipes 14 are introduced into the supply connectors 9, while being introduced into the supply connectors 9, or after being introduced into the supply connectors 9.

The steps of introducing treatment liquids, the treatment time and the removal of the treatment liquid may be repeated, if necessary, according to a predefined treatment scheme. In case only one treatment step is required, the treatment cycle may be completed after only one treatment step.

After the treatment cycle is completed, the grippers remove the contact lenses 2 from the cavities 5 of the treatment containers 1 in the container plate 13 and transport the contact lenses 2 to the next station in the manufacturing line. For example, the next step may be the inspection of the contact lenses 2. The container plate 13 may be inspected between two treatment cycles in order to check whether all treatment containers 1 in the container plate 13 are empty so that the container plate 13 is ready for the next cycle.

In order to achieve a constant high quality, each treatment container 1 of the container plate 13 may be rinsed with deionized water between two treatment cycles.

The treatment container according to the invention can be used in a fully automated production processes for ophthalmic lenses, in particular lenses such as (soft) contact lenses or intraocular lenses. The treatment container is simple in construction and capable of being reproducibly manufactured in mass production processes, for example by injection molding, by a deep drawing process or by a process of milling a block.

While embodiments of the invention have been described with the aid of the drawings, various changes, modifications, and alternatives are conceivable without departing from the teaching underlying the invention. Therefore, the invention is not intended to be limited to the described embodiments but rather is defined by the scope of the appended claims.

1. Treatment container (1) for accommodating an ophthalmic lens (2), in particular a contact lens such as a hard or soft contact lens, or an intraocular lens, the treatment container (1) comprising
a bottom (3),
a side wall (4) surrounding the bottom (3) and together with the bottom (3) forming a cavity (5) for accommodating the ophthalmic lens (2),
a container opening (6) arranged opposite to the bottom (3), and
a fluid opening (7) arranged in the bottom (3) or in the side wall (4) of the treatment container (1), the fluid opening (7) being in fluid communication with the cavity (5),
the treatment container (1) further comprising
a fluid conveyance channel (8) and
a supply connector (9),
wherein the fluid conveyance channel (8) is connected to the fluid opening (7) so as to be in fluid communication with the fluid opening (7), and wherein the fluid conveyance channel (8) is further connected to the supply connector (9) so as to be in fluid communication with the supply connector (9), to allow for introducing a treatment liquid into the cavity (5) or for removing a treatment liquid from the cavity (5) through the supply connector (9), the conveyance channel (8) and the fluid opening (7).

2. Treatment container (1) according to claim 1, wherein a surface of the bottom (3) that faces towards the cavity (5) is concavely shaped.

3. Treatment container (1) according to claim 1 or claim 2,
wherein the treatment container (1) has a rim (10) at one end of the side wall (4) arranged at the container opening (6), the rim (10) being arranged in a plane and surrounding the container opening (6),
and wherein the fluid opening (7) is arranged, at least in part, in the bottom (3) of the container at a location in the cavity (5) which is arranged most distally from the plane in which the rim (10) surrounding the container opening (6) is arranged.

4. Treatment container (1) according to any one of claims 1 to 3, wherein a surface of the bottom (3) that faces towards the cavity (5) comprises a structure (9) facing the interior of the cavity (5), to allow for uniform wetting of the ophthalmic lens (2).

5. Treatment container (1) according to claim 4, wherein the structure comprises one or more grooves (11) arranged in the bottom surface, in particular four grooves arranged at 90° or eight grooves arranged at 45° relative to one another when viewed in a circumferential direction of the container, and meeting at an intersection (12) located centrally in the bottom surface.

6. Treatment container (1) according to claim 5, wherein the fluid opening (7) is connected, at least in part, to one of the one or more grooves (11) arranged in the bottom surface so as to be in fluid communication with the one of the one or more grooves (11), to allow for introduction and/or removal of the treatment liquid.

7. Container plate (13) comprising a plurality of treatment containers (1) according to any one of claims 1 to 6.

8. Container plate (13) according to claim 7, wherein the supply connectors (9) of two or more treatment containers (1) of the plurality of treatment containers (1) together form a common supply connector (9), and wherein the fluid openings (7) of the two or more treatment containers (1) are in fluid communication with the common supply connector (9) via the conveyance channels (8) of the two or more treatment containers (1), for concurrently introducing a treatment liquid into or concurrently removing a treatment liquid from the two or more treatment containers (1) which are in fluid communication with the common supply connector (9).

9. Container plate (13) according to claim 8, wherein the common supply connector (9) is configured to receive a supply pipe, in particular a suction pipe (14) from a liquid aspirator.

10. Container plate (13) according to claim 9, wherein the container plate (13) has a plate bottom (15) and a plate top surface (16), and wherein each of the supply connectors (9) has a supply pipe receiving opening (17) which is arranged at the plate top surface (16) opposite to the plate bottom (15).

11. Container plate (13) according to any one of claims 7 to 10, wherein the plurality of treatment containers (1) are integrally formed with the container plate (13), so that the container plate (13) including the plurality of treatment containers (1) is made from one piece, thus forming a monolithic container plate.

12. Container plate (13) according to claim 11, wherein the plurality of treatment containers (1), the liquid conveyance channels (8) and the supply connectors (9) are formed during injection molding of the monolithic container plate (13), by a deep drawing process or by milling a bulk plate.

13. Container tray (18) comprising a container plate (13) according to any one of claims 7 to 12, and a container plate frame (19) holding the container plate (13) and having fixtures adapted to cooperate with corresponding fixtures of a production facility.

14. Method for treatment of an ophthalmic lens (2), in particular a contact lens such as a hard or soft contact lens, or an intraocular lens, with a treatment liquid, the method comprising the steps of:
providing a container plate (13) according to any one of claims 7 to 13,
introducing ophthalmic lenses (2) into the cavities (5) of the treatment containers (1) of the container plate (13),
filling the treatment containers (1) with a treatment liquid either through the container openings (6) or through the fluid openings (7),
removing the treatment liquid through the fluid openings (7), either after a predetermined time during which the ophthalmic lenses (2) are exposed to the treatment liquid or during filling the treatment containers (1) with the treatment liquid,
optionally repeating the steps of filling the treatment containers (1) with the treatment liquid and removing the treatment liquid in accordance with a predetermined treatment scheme to complete a treatment cycle, and
removing the ophthalmic lenses (2) from the cavities (5) of the treatment containers (1).

15. Method according to claim 14, wherein the treatment containers (1) are rinsed with a rinsing liquid between two subsequent treatment cycles.
